(19) United States
(12) Patent Application Publication
(10) Pub. No.: US 2012/0028093 A1
(43) Pub. Date: Feb. 2, 2012

(54) IONIC CONDUCTIVE SIDE-CHAIN-TYPE POLYMER ELECTROLYTE, PRECURSOR THEREOF, AND LITHIUM SECONDARY BATTERY

(76) Inventors: Akira SATOU, Hitachiomiya (JP); Shin NISHIMURA, Hitachi (JP)

(21) Appl. No.: 13/271,500

(22) Filed: Oct. 12, 2011

Related U.S. Application Data
(62) Division of application No. 11/485,446, filed on Jul. 13, 2006.

(30) Foreign Application Priority Data
Jul. 15, 2005 (JP) 2005-207195

(51) Int. Cl. H01M 10/0565; C07C 69/96; H01M 10/0587; B01J 39/20

(52) U.S. Cl. 429/94; 521/31; 558/260; 429/314; 429/189

(57) ABSTRACT

This invention provides a side-chain-type polymer electrolyte exhibiting high ionic conductivity and a lithium secondary battery using the same. Such side-chain-type polymer electrolyte comprises a polymer structural unit represented by formula (1):

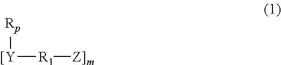

wherein Rₚ represents an organic group obtained via polymerization of monomer compounds containing polymerizable unsaturated linkages or a polymerized organic group containing C, H, N, and O; m represents a value smaller than the polymerization degree of Rₚ; Y represents an organic group that binds to Rₚ; R₁ represents a C₁₋₁₀ alkylene group that allows Y to bind to Z; and Z represents a functional group having coordination ability with respect to a cation, provided that Z forms a coordination bond with a cation,

wherein the polymer electrolyte has composition wherein a cation is added to a polymer having a side chain consisting of R₁ and Z binding through Y to a polymer main chain consisting of Rₚ.

The present application is a divisional of U.S. application Ser. No. 11/485,446 filed Jul. 13, 2006, and claims priority from Japanese Application No. 2005-207195 filed on Jul. 15, 2005, the contents of each of which are hereby incorporated by reference into this application.

### BACKGROUND OF THE INVENTION

1. Technical Field

The present invention relates to an ionic conductive polymer electrolyte, a precursor thereof, and a lithium secondary battery.

2. Description of Related Art

Advances in electronics have allowed the performances of electronic devices to be enhanced, and electronic devices have been miniaturized and made portable. Accordingly, secondary batteries with high energy density have been needed as power sources for such devices. In response to such need, nonaqueous electrolyte system secondary batteries with significantly enhanced energy density, i.e., lithium ion secondary batteries with organic electrolytic solution (hereafter simply referred to as “lithium batteries”), have been developed, and they have become widely prevalent in recent years. Lithium batteries use, for example, lithium metal complex oxides such as lithium-cobalt complex oxides as positive electrode active materials. They primarily use as their negative electrode active materials multilayered carbon materials capable of intercalating lithium ions in the layered structure (formation of lithium intercalation compounds) and deintercalating lithium ions out of the layered structure.

Lithium batteries use a combustible organic electrolytic solution. Thus, securing of safety in the case of overuse, such as overcharge or over-discharge, is becoming difficult with the enhancement in energy density of the batteries. Accordingly, lithium polymer batteries in which the combustible organic electrolytic solution has been replaced with a solid lithium-ionic conductive polymer were developed.

A mechanism of an ionic conductive polymer for conducting ions that has heretofore been examined is known to occur in conjunction with the motion of a polymer molecular chain. A representative example of a lithium ionic conductive polymer is poly(ethylene oxide). For example, an application possibility of poly(ethylene oxide) as a lithium ionic conductive polymer electrolyte is pointed out by Armand et al. (Non-Patent Document 1). Various improvements of poly(ethylene oxide) have been performed and other polymers are being studied. An ionic conductive polymer having the highest ionic conductivity is copolymer of branched ethylene oxide and propylene oxide as described in a publication of patent application (Patent Document 1). The ionic conductivity is approximately 10⁻⁴ Scm⁻¹. Ionic conductivity is governed by mobility of the molecular chain and by the motion of a molecular chain having high activation energy, which is required for segmental motion. Thus, ionic conductivity at room temperature is approximately 10⁻⁴ Scm⁻¹, but it becomes significantly lower as the temperature drops.

In order to reduce the activation energy of the molecular chain motion, which is an ion-conducting mechanism, the present inventors conceived of aligning a side chain having an ionic conductive functional group to a polymer main chain.

An organic group having a functional group, which is a ligand coordinated to a lithium ion, is bound to a polymer main chain as a polymer side chain, and the molecular chain of the side chain is considerably shorter than that of the polymer main chain. Accordingly, the mobility of the polymer side chain is higher than that of the polymer main chain, which enables the reduction in the activation energy. By the motion of the side chain, a lithium ion is transported to a similar functional group of the adjacent side chain, and ionic conduction then takes place. This ion-conducting mechanism realizes the preparation of a polymer electrolyte having excellent temperature dependence.
Patent Document 1: JP Patent Publication (Unexamined) No. 2000-123632
Non-Patent Document 1: “Fast Ion Transport in Solids,” p. 131, Elsevier, N.Y., 1979

### SUMMARY OF THE INVENTION

A mechanism of an ionic conductive polymer for conducting ions that has heretofore been examined is known to occur in conjunction with the motion of a polymer molecular chain. Specifically, a functional group of the molecular chain having coordination ability is coordinated to a lithium ion in a solid, and a lithium ion migrates via transition of such coordination to another ligand by the motion of the molecular chain. Accordingly, ionic conductivity is governed by mobility of the molecular chain and by motion having high activation energy, which is required for segmental motion, such as dihedral angular motion of the main chain that takes place upon morphological change of the molecular chain. Thus, ionic conductivity may also be simultaneously and disadvantageously lowered under a low temperature where molecular motions are suppressed.

An organic group having a functional group, which is a ligand coordinated to a lithium ion, is bound to a polymer main chain as a polymer side chain that is considerably shorter than the polymer main chain, in order to enhance the mobility of the organic group than that of the polymer main chain. Motion of the side chain can reduce the activation energy generated upon conduction of a lithium ion to a similar functional group of the adjacent side chain. This can realize the preparation of a polymer electrolyte having excellent temperature dependence of the ionic conductivity.

### BRIEF DESCRIPTION OF THE DRAWING

FIG. 1 shows a cross section of a lithium secondary battery according to Example 3.

Hereafter, the embodiments of the present invention are described.

### DETAILED DESCRIPTION OF THE PREFERRED EMBODIMENTS

According to an embodiment of the present invention, a cationic conductor is a side-chain-type polymer electrolyte comprising a polymer structural unit represented by formula (1):

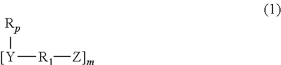

wherein Rₚ represents an organic group obtained via polymerization of a compound containing polymerizable unsaturated linkages or a polymerized organic group containing C, H, N, and O; m represents a value smaller than the polymerization degree of Rₚ; Y represents an organic group that binds to Rₚ; R₁ represents a C₁₋₁₀ alkylene group that allows Y to bind to Z; and Z represents a functional group having coordination ability with respect to a cation, provided that Z forms a coordination bond with a cation,

wherein the polymer electrolyte has composition wherein a cation is added to a polymer having a side chain consisting of R₁ and Z binding through Y to a polymer main chain consisting of Rₚ.

In the case of the cationic conductor of this embodiment, a polymer side chain comprising organic group R₁ and organic group Z is bound to polymer main chain Rₚ, and the polymer side chain moves with the aid of thermal vibration. The compound of this embodiment exhibits cationic conductivity via easy migration and exchange of cations coordinated to functional group Z between adjacent organic groups Zs.

It is important that mobility of the polymer side chain comprising organic groups R₁ and Z be high. A component of the polymer side chain is not limited to functional groups such as organic group R₁ and Z.

According to an embodiment of the present invention, a cationic conductor is a side-chain-type polymer electrolyte having a polymer structural unit represented by formula (2) having a carbonate group corresponding to Z in formula (1):

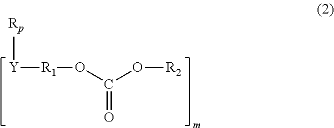

wherein Rₚ represents an organic group obtained via polymerization of monomer compounds containing polymerizable unsaturated linkages or a polymerized organic group containing C, H, N, and O; m represents a value smaller than the polymerization degree of Rₚ; Y represents an organic group that binds to Rₚ; and R₁ represents a C₁₋₁₀ alkylene group that allows Y to bind to a carbonate group, provided that the carbonate group is a functional group having coordination ability with respect to a cation and forms a coordination bond with a cation,

wherein the polymer electrolyte has composition wherein a cation is added to a polymer having a side chain consisting of R₁ and the carbonate group binding through Y to a polymer main chain consisting of Rₚ.

When Y in formula (2) is represented by formula (3), a side-chain-type polymer electrolyte represented by formula (10) is obtained.

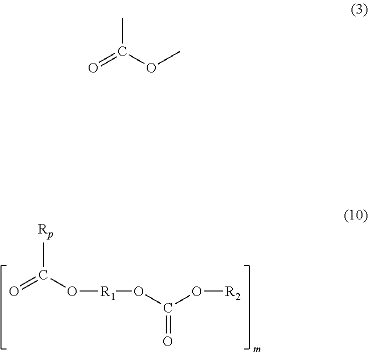

When Y in formula (2) is represented by formula (4), a side-chain-type polymer electrolyte represented by formula (11) is obtained.

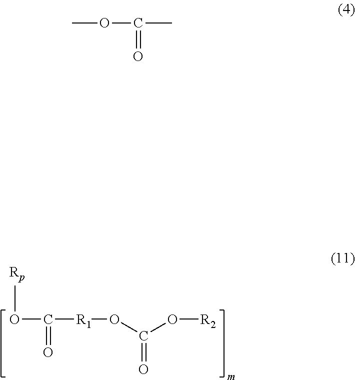

When Y in formula (2) is represented by formula (5), a side-chain-type polymer electrolyte represented by formula (12) is obtained.

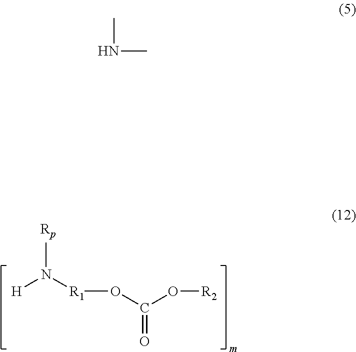

When Y in formula (2) is represented by formula (6), a side-chain-type polymer electrolyte represented by formula (13) is obtained.

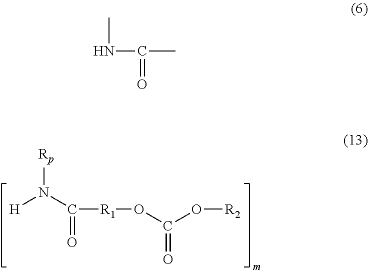

When Y in formula (1) is represented by formula (7), a side-chain-type polymer electrolyte represented by formula (14) is obtained.

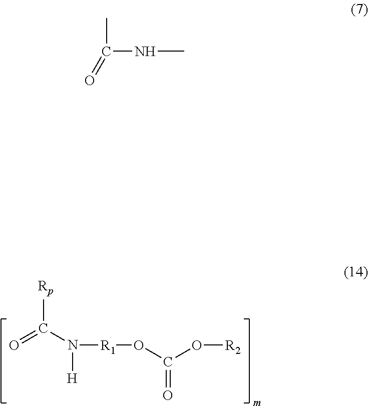

When Y does not exist in formula (2), a polymer represented by formula (8) is obtained.

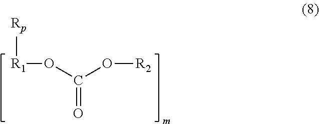

A monomer, which is a precursor of the polymer synthesis, is represented by formula (9).

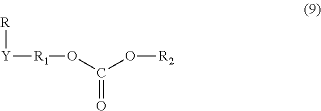

When an ion is coordinated to functional group Z and it migrates to an adjacent functional group by the motion of the functional group, ionic conduction takes place. When the functional group Z has potent coordination ability, accordingly, it becomes difficult to release an ion while coordination is maintained. This may inhibit the ionic conduction.

In this embodiment, organic group Z has functional Z that can coordinate to a cation. An example thereof is a carbonate group (—O—C(═O)—OR, where R=an alkyl group). An enlarged alkyl group inhibits the mobility of the side chain or affects the ion transmission between adjacent functional groups. This may lower the conductivity.

When functional group Z is methoxy (—OCH₃), an organic group can be an alkoxy phenyl group such as a methoxy phenyl or dimethoxy phenyl group. A methoxy or ethoxy group can be used as an alkoxy group (—OR, where R=an alkyl group). An alkylthio group that is prepared by substituting an oxygen atom with a sulfur atom in an alkoxy group may also be used. Also, functional group Z can also be used in the form of ester (—O—C(═O)—R, —C(═O)O—R), an amino group (—NR₁R₂), or an acyl group (—C(═O)—R).

In this embodiment, organic group Rₚ is not particularly limited, and a variety of organic groups, such as a saturated hydrocarbon compound, an unsaturated hydrocarbon compound, or an aromatic hydrocarbon compound, can be employed. Such organic group is not limited to a hydrocarbon compound, and an organic group may contain elements, such as nitrogen, sulfur, or oxygen. Alternatively, part of such organic group may be substituted by halogen. The molecular weight thereof is not limited, and low-molecular-weight to high-molecular-weight compounds can be employed. A high-molecular-weight compound may be a polymer of low-molecular-weight monomers.

When organic group Rₚ is an unsaturated hydrocarbon polymer, a means of addition polymerization can be employed. Butyl lithium, azobisisobutyronitrile, or peroxides such as benzoyl peroxide or PV t-hexyl peroxypivalate can be used as an initiator for polymerization where a polymer is generated.

A means for polymerizing a polymer represented by organic group Rₚ is not particularly limited. For example, addition polymerization, polyaddition, or polycondensation can be employed without particular limitation.

In this embodiment, lithium is employed as a cation. Alkali metal ions such as sodium or potassium, alkaline earth metals such as magnesium, or a hydrogen ion can also be used. Among them, lithium ions are most preferable.

Lithium salts can also be used as lithium ion sources. Examples of lithium salts include LiN(CF₃CF₂SO₂)₂, LiN(CF₃SO₂)₂, LiClO₄, LiPF₆, LiBF₄, and LiAsF₆, and they can be used solely or in combinations of two or more. LiN(CF₃CF₂SO₂)₂ is particularly preferable. Preferably, at least 1 equivalent of lithium ions is added relative to one organic group Z, which is involved with lithium conduction, in terms of a molar proportion.

### Example 1

A method 1 for synthesizing a cationic conductor represented by formula (8) is described. Allyl methyl carbonate (50 g) is dissolved in 0.5 dm³ of tetrahydrofuran, 0.25 g of AIBN is added thereto, and the mixture is stirred at 70° C. to obtain a polymer. The resulting polymer (1 g) and 1 g of LiN(CF₃CF₂SO₂)₂ are dissolved in 20 ml of N-methylpyrrolidone, the resulting solution is cast on a poly(tetrafluoroethylene) sheet, the sheet is subjected to vacuum drying at 80° C., and a cast film having a thickness of 100 μm is prepared.

This cast film is inserted between stainless (SUS 304) electrodes with diameters of 15 mm to prepare a test cell. An amplitude voltage of 10 mV is applied to this cell at room temperature to measure a.c. impedance. The frequency range is between 1 Hz and 1 MHz. Based on the reciprocal of the bulk ohmic value obtained by the measurement of a.c. impedance, ionic conductivity is determined. Ionic conductivity is deduced to be approximately 5×10⁻⁵ Scm⁻¹ at room temperature.

### Example 2

A.c. impedance was measured in order to examine the temperature dependence of the ionic conductivity using the test cell prepared in Example 1. The test cell was allowed to stand in a thermostat maintained at the given temperature level for 30 minutes, and the measurement was carried out in a manner such that the cell was set in the thermostat. Ionic conductivity was determined in the same manner as in Comparative Example 1. The activation energy of the ionic conduction, which was calculated based on the correlation between ionic conductivity and temperature, was deduced to be 5 kJ/mol, which is smaller than that obtained in Comparative Example 2 below. A polymer electrolyte having excellent temperature dependence can be thus obtained.

### Example 3

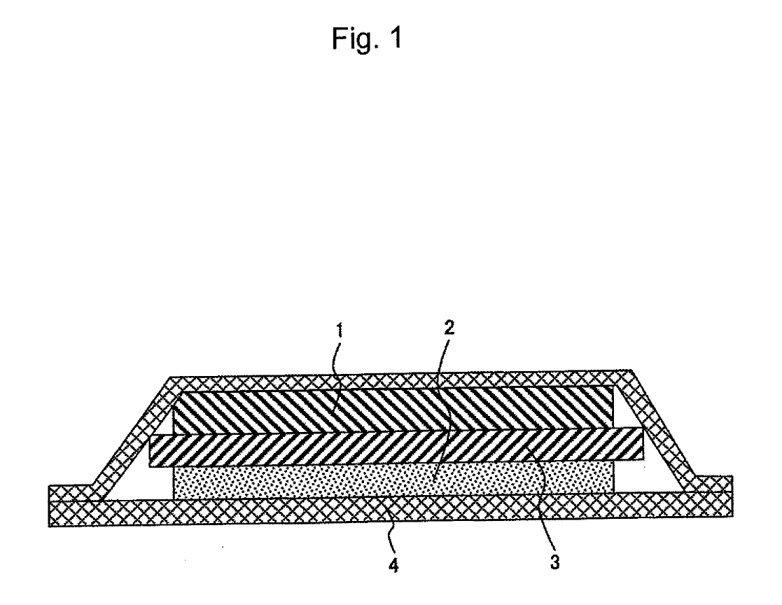

FIG. 1 shows a cross section of a lithium battery using a cationic conductive polymer electrolyte according to an embodiment of the present invention.

A lithium ionic conductive polymer electrolyte of the present example is a complex of a polymer and a lithium salt. Such electrolyte can be obtained by dissolving a monomer having an organic group that affects ionic conduction and a lithium salt in an organic solvent, subjecting the resulting solution to polymerization, and then removing an organic solvent. Alternatively, a polymer having an organic group that affects ionic conduction is dissolved in an organic solvent, and an organic solvent is then removed therefrom. Thus, a lithium ionic conductive polymer electrolyte can also be obtained.

A polymer electrolyte is prepared in the form of a sheet when it is used as an electrolyte for a lithium battery and is made to function as a separator between positive and negative electrodes. Such sheet-like polymer electrolyte can be obtained by dissolving a polymer having an organic group that affects ionic conduction and a lithium salt in an organic solvent, subjecting the resulting solution to addition polymerization by heating, and removing an organic solvent by evaporation. Alternatively, a polymer having an organic group that affect ionic conduction is dissolved in an organic solvent, a lithium salt is added thereto, the resultant is cast on a poly(tetrafluoroethylene) sheet, and an organic solvent is then removed by evaporation. Thus, a polymer electrolyte of interest can also be obtained.

Examples of an organic solvent that dissolves a polymer electrolyte and a lithium salt include N-methylpyrrolidone, dimethylformamide, toluene, propylene carbonate, and γ-butyrolactone, which thoroughly dissolve the lithium salt but do not react with the polymer.

A positive electrode active material that reversibly intercalates and deintercalates lithium may be at least one of the following: a layered compound such as a lithium cobalt oxide (LiCoO₂) or lithium nickel oxide (LiNiO₂); a layered compound in which at least one kind of transition metal has been substituted; a lithium manganese oxide (Li₁₊ₓMn₂₋ₓO₄, where X=0 to 0.33; Li₁₊ₓMn_{2-X-Y}M_{Y}O₄, where M is at least one member selected from the group of metals consisting of Ni, Co, Cr, Cu, Fe, Al, and Mg, X=0 to 0.33, and Y=0 to 1.0, and 2-X-Y>0; LiMnO₃, LiMn₂O₃, LiMnO₂, or LiMn₂₋ₓMₓO₂, where M is at least one member selected from the group of metals consisting of Co, Ni, Fe, Cr, Zn, and Ta, and X=0.01 to 0.1; Li₂Mn₃MO₈, where M is at least one member selected from the group of metals consisting of Fe, Co, Ni, Cu, and Zn); a copper-lithium oxide (Li₂CuO₂); an oxide of vanadium such as LiV₃O₈, LiFe₃O₄, V₂O₅, V₆O₁₂, VSe, or Cu₂V₂O₇; a disulphide compound; a mixture containing Fe₂(MoO₄)₃ etc; polyaniline; polypyrrole; and polythiophene.

A negative electrode active material that reversibly intercalates and deintercalates lithium include: an easily graphitizable material obtained from natural graphite, petroleum coke, or coal pitch coke that has been subjected to heat treatment at high temperatures of 2500° C. or higher; mesophase carbon or amorphous carbon; carbon fiber; a lithium metal; a metal that alloys with lithium; or a carbon particle carrying a metal on the surface thereof. Examples thereof include metals or alloys selected from the group consisting of lithium, aluminum, tin, silicon, indium, gallium, and magnesium. These metals or their oxides may be utilized for the negative electrode active materials.

A polymer battery of the present example comprises a positive electrode prepared from the aforementioned positive electrode active material and a negative electrode prepared from the aforementioned negative electrode active material separated by a sheet-like polymer electrolyte. Also, positive and negative electrodes containing polymer electrolytes can be prepared in order to enhance adhesion between a positive or negative electrode active material and a polymer electrolyte. In such a case, a monomer having an organic group that affects ionic conduction and a lithium salt are dissolved in an organic solvent, the resulting solution is cast on the positive and negative electrodes, and heat polymerization is then carried out. Alternatively, a lithium salt and a copolymer comprising an organic group that affects ionic conduction are dissolved in an organic solvent, the resulting solution is cast on the electrodes, and an organic solvent is then removed. Thus, such electrodes can be obtained. The thus-obtained positive and negative electrodes may be bound to each other to obtain a polymer battery.

A lithium battery with the polymer electrolyte is suitably mounted on electric equipment as shown below. For example, such polymer electrolyte may be utilized for lithium secondary batteries as the electric power supplies for: electric automobiles; electric bicycles; personal computers; cellular phones; digital cameras; camcorders; portable minidisc players; personal digital assistants; wrist watches; radios; electronic personal organizers; electric tools; vacuum cleaners; toys; elevators; robots for emergency purposes; walking-aid machines for healthcare purposes; wheelchairs for healthcare purposes; moving beds for healthcare purposes; emergency electric supplies; load conditioners; and electric power storage systems. Since no electrolytic fluid is used, it is expected that the safety level is enhanced and need of a protection circuit is eliminated. Thus, lithium secondary batteries can be used as rechargeable batteries for household use, the size thereof can be enlarged, and thus, they are suitable as dispersed power sources for household and regional use. The performance level can be maintained at low temperature no different from that at room temperature, fluid does not leak at high temperatures, and thus, the batteries can be used in a wide temperature range. Accordingly, they may also be utilized as the power supplies for military, space-exploration, or emergency purposes, as well as for consumer applications.

### Comparative Example 1

A copolymer (37 g) of ethylene oxide (80% by mole) and 2-(2-methoxyethoxy)ethyl glycidyl ether (20% by mole) was mixed with 6.6 g of LiPF₆ as an electrolytic salt, and the mixture was dissolved in acetonitrile to prepare a solution. The resulting solution was cast on a poly(tetrafluoroethylene) sheet, the sheet was subjected to vacuum drying at 80° C., and a cast film having a thickness of 100 μm was prepared. This cast film was inserted between stainless (SUS 304) electrodes with diameters of 15 mm to prepare a test cell. An amplitude voltage of 10 mV was applied to this cell at room temperature to measure a.c. impedance. The frequency range was between 1 Hz and 1 MHz. Based on the reciprocal of the bulk ohmic value obtained by the measurement of a.c. impedance, ionic conductivity was determined. Ionic conductivity was found to be 5×10⁻⁵ Scm⁻¹.

### Comparative Example 2

A.c. impedance was measured in order to examine the temperature dependence of the ionic conductivity using the test cell prepared in Comparative Example 1. The test cell was allowed to stand in a thermostat maintained at the given temperature level for 30 minutes, and the measurement was carried out in a manner such that the cell was set in the thermostat. Ionic conductivity was determined in the same manner as in Comparative Example 1. The activation energy of the ionic conduction, which was calculated based on the correlation between ionic conductivity and temperature, was found to be 40 kJ/mol.

### EFFECTS OF THE INVENTION

The present invention can provide an electrolyte having excellent temperature dependence and a lithium secondary battery.

1. A side-chain-type polymer electrolyte having a polymer structural unit represented by formula (8): 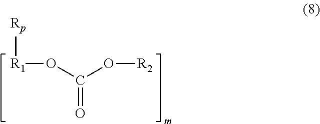
wherein Rₚ represents an organic group obtained via polymerization of a compound containing polymerizable unsaturated linkages or a polymerized organic group containing C, H, N, and O; m represents a value smaller than the polymerization degree of Rₚ; and R₁ represents a C₁₋₁₀ alkylene group that allows Rₚ to bind to a carbonate group, provided that the carbonate group is a functional group having coordination ability with respect to a cation and forms a coordination bond with a cation,
wherein said polymer electrolyte has composition wherein a cation is added to a polymer having a side chain consisting of R₁ and a carbonate group binding through R₁ to a polymer main chain consisting of Rₚ.

2. The side-chain-type polymer electrolyte according to claim 1, wherein R₁ represents not more than 8 methylene groups.

3. The side-chain-type polymer electrolyte according to claim 1, wherein R₂ represents a methyl or ethyl group.

4. A precursor of the side-chain-type polymer electrolyte according to claim 1, wherein the precursor is represented by formula (9), 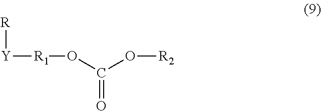
wherein R represents an organic group having polymerizable unsaturated linkages; Y does not exist; R₁ represents a C₁₋₁₀ alkylene group that allows R to bind to a carbonate group; and R₂ represents an organic group that binds to an end of the carbonate group, provided that the carbonate group is a functional group having coordination ability with respect to a cation and forms a coordination bond with a cation.

5. A lithium secondary battery comprising a positive electrode having a positive electrode active material that can intercalate and deintercalate lithium and a negative electrode having a negative electrode active material that can intercalate and deintercalate lithium that are rolled or laminated via an interposing polymer electrolyte, wherein the polymer electrolyte comprises the side-chain-type polymer electrolyte according to claim 1, the side-chain-type polymer electrolyte functioning as a cationic conductor.

6. A lithium secondary battery comprising a positive electrode having a positive electrode active material that can intercalate and deintercalate lithium and a negative electrode having a negative electrode active material that can intercalate and deintercalate lithium that are rolled or laminated via an interposing polymer electrolyte, wherein the polymer electrolyte comprises the side-chain-type polymer electrolyte according to claim 2, the side-chain-type polymer electrolyte functioning as a cationic conductor.

7. A lithium secondary battery comprising a positive electrode having a positive electrode active material that can intercalate and deintercalate lithium and a negative electrode having a negative electrode active material that can intercalate and deintercalate lithium that are rolled or laminated via an interposing polymer electrolyte, wherein the polymer electrolyte comprises the side-chain-type polymer electrolyte according to claim 3, the side-chain-type polymer electrolyte functioning as a cationic conductor.

8. A lithium secondary battery comprising a positive electrode having a positive electrode active material that can intercalate and deintercalate lithium and a negative electrode having a negative electrode active material that can intercalate and deintercalate lithium that are separated by the precursor of the side-chain-type polymer electrolyte according to claim 4.
